(19) United States
(12) Patent Application Publication
(10) Pub. No.: US 2011/0082087 A1
(43) Pub. Date: Apr. 7, 2011

(54) COMPOUNDS FOR TREATING PAIN

(75) Inventors: Marta Maria Batista Ribeiro, Lisboa (PT); Miguel Augusto Rico Botas Castanho, Lisboa (PT); Christophe Francois Aime Roca, Lisboa (PT); Helena Margarida Moreira de Oliveira Vieira, Lisboa (PT); Jose Manuel Bernardo de Sousa, Lisboa (PT); Marta Isabel Heitor Cerejo, Lisboa (PT); Patricia Ramalhete Mendes da Silva Calado, Lisboa (PT); Sukalyan Chatterjee, Lisboa (PT); Eduard Bardaji Rodriguez, Girona (ES); Montserrat Heras Corominas, Girona (ES); Isaura Ferreira Tavares, Porto (PT); Marta Sofia Carvalho Teixeira Pinto, Porto (PT); Ana Dulce Ascensao Correia, Lisboa (PT); Manuel Nuno de Sousa Simoes de Mel, Lisboa (PT)

(73) Assignee: Edificio ICAT, Campo Grande, Lisboa (PT)

(21) Appl. No.: 12/935,891

(22) PCT Filed: Apr. 1, 2009
(86) PCT No.: PCT/PT2009/000019

§ 371 Date: Dec. 20, 2010

(30) Foreign Application Priority Data
Apr. 1, 2008 (GB) 0805912.3

(51) Int. Cl. A61K 38/05; C07C 237/22; A61P 29/00

(52) U.S. Cl. 514/18.3; 514/21.91; 564/157; 564/153

(57) ABSTRACT

The present invention relates to a compound of formula (I)

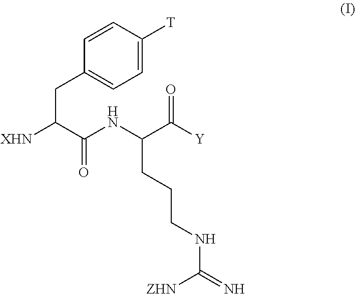

wherein X is hydrogen, R¹, R¹C(O), R¹CO₂, or a COX2 inhibitor,
wherein R¹ is C₁₋₂₀ alkyl, aryl, arylalkyl, alkyloxy or arylalkyloxy;
wherein Y is OR², NHR³N(R³)₂, or a COX inhibitor;
wherein R² is hydrogen or C₁₋₂₀ alkyl and each R³ is independently hydrogen or a C₁₋₄ alkyl;
wherein T is OR⁴, NHR⁵N(R⁵)₂, or a COX inhibitor
wherein R⁴ is hydrogen or C₁₋₂₀ alkyl and each R⁵ is independently hydrogen or a C₁₋₄ alkyl;
wherein Z is hydrogen, R⁶, R⁶C(O), R⁶CO₂, or a COX2 inhibitor;
wherein R⁶ is C₁₋₂₀ alkyl, aryl, arylalkyl, alkyloxy or arylalkyloxy;
with the proviso wherein when X and Z are hydrogen and T is OH, Y is not OH; for use in the prevention and/or treatment of pain wherein said compound is provided for systemic administration.

The present invention relates to the provision of derivatives of L-Tyrosyl-L-Arginine for use in the treatment of pain, said derivative being administered systematically.

Kyotorphin (L-Tyrosyl-L-Arginine; KTP) was first discovered in 1979 and reported as an endogenous analgesic agent in the brain. However, attempts to utilise Kyotorphin as an analgesic have been unsuccessful due to the inability of Kyotorphin to cross the Blood-brain-barrier (BBB). In particular, attempts to modify Kyotorphin to overcome this limitation, including derivatisation with hydrophobic groups, have not addressed this problem.

The present invention provides derivatised forms of Kyotorphin, which can be administered systematically for use as an analgesic.

The first aspect of the invention relates to a compound of formula (I)

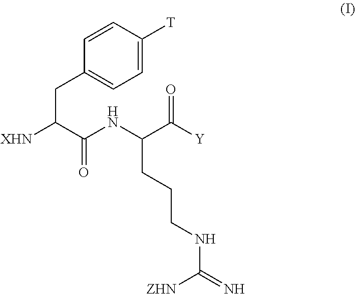

wherein X is hydrogen, R¹, R¹C(O), R¹CO₂, or a COX2 inhibitor,
wherein R¹ is C₁₋₂₀ alkyl, aryl, arylalkyl, alkyloxy or arylalkyloxy,
wherein Y is OR², NHR³, N(R³)₂, or a COX2 inhibitor
wherein R² is hydrogen or C₁₋₂₀ alkyl and each R³ is independently hydrogen or a C₁₋₄ alkyl;
wherein T is OR⁴, NHR⁵, N(R⁵)₂, or a COX2 inhibitor
wherein R⁴ is hydrogen or C₁₋₂₀ alkyl and each R⁵ is independently hydrogen or a C₁₋₄ alkyl;
wherein Z is hydrogen, R⁶, R⁶C(O), R⁶CO₂, or a COX2 inhibitor
wherein R⁶ is C₁₋₂₀ alkyl, aryl, arylalkyl, alkyloxy or arylalkyloxy, with the proviso wherein when X and Z are hydrogen and T is OH, Y is not OH; for use in the prevention and/or treatment of pain wherein said compound is provided for systemic administration.

It will be appreciated that the compound of formula (I) is a derivatised form of kyotorphin (L-Tyrosyl-L-Arginine). Preferably, the invention relates to a compound of formula (I) wherein X is hydrogen, or a COX2 inhibitor, Y is hydroxy, NH₂ or a COX inhibitor, Z is hydrogen and T is OH; with the proviso wherein when X and Z are hydrogen and T is OH, Y is not OH.

For the purposes of this invention, the compound of formula (I) can comprise a COX2 inhibitor at any of positions X, Z, T or Y. Preferably only one of positions X, Z, T or Y contains a COX2 inhibitor. The COX2 inhibitor can be independently selected from ibuprofen, acetylsalicilic acid, meloxicam, valdecoxib, celecobix or refocobix. Preferably, the COX2 inhibitor is ibuprofen or acetylsalicilic acid.

In a particular embodiment, the invention relates to a compound of formula (I)

wherein X and Z are hydrogen, T is hydroxyl and Y is NH₂ (in particular L-Tyr-D-Arg-NH₂, D-Tyr-D-Arg-NH₂, or D-Tyr-L-Arg-NH₂) or

wherein X is ibuprofen, Z is hydrogen, T is hydroxyl and Y is NH₂ (in particular ibuprofen-L-Tyr-L-Arg-NH₂ or ibuprofen-D-Tyr-L-Arg-NH₂) or

wherein X is ibuprofen, Z is hydrogen and T and Y are hydroxyl (in particular ibuprofen-L-Tyr-L-Arg-OH) or

wherein X is methyl, Z is hydrogen, T is hydroxyl and Y is NH₂ (in particular methyl-L-Tyr-L-Arg-NH₂, methyl-L-Tyr-D-Arg-NH₂, methyl-D-Tyr-L-Arg-NH₂ or methyl-D-Tyr-D-Arg-NH₂).

For the purposes of this invention, the compound of formula (I) is provided for systemic administration and is not provided for topical application or administration. The present invention therefore particularly relates to the provision of the compound of formula (I) for enteral or parenteral administration. Enteral routes of administration include oral (including inhalation), mucosal (including buccal, sublingual, nasal), vaginal or rectal. More particularly, the compound of formula (I) can be administered by intravenous administration, intraarterial administration, intramuscular administration, intracardiac administration, subcutaneous administration, intraosseious infusion, intradermal administration, intrathecal administration, intraperitoneal administration, tranmucosal administration, epidural administration and/or by intravitreal administration.

The first aspect of the invention is provided for the prevention and/or treatment of pain. For the purposes of this invention, the term pain includes acute or chronic pain and includes neuropathic pain. The pain may be caused by a disease such as cancer or by a trauma such as an injury to the body (i.e. an injury to the back, neck, head, leg, arm etc) or as a result of surgery, or may have a physiological cause, such as migraine.

The administration of the compound of formula (I) may result in the complete amelioration of the pain. Alternatively, the administration of the compound of formula (I) may reduce the severity of the pain. In a preferred embodiment, the pain is reduced to a level which is acceptable or bearable to the patient.

For the purposes of the present invention, a “C₁₋₂₀ alkyl group” as used herein is an alkyl group that is a straight or branched chain with 1 to 20 carbons. The alkyl group therefore has 1, 2, 3, 4, 5, 6, 7, 8, 9, 10, 11, 12, 13, 14, 15, 16, 17, 18, 19 or 20 carbon atoms. The alkyl group can be optionally saturated at one or more positions along the carbon chain. The alkyl group can be hydroxylated at one or more positions along its length. Preferably, the alkyl group has from 1 to 10 carbon atoms, more specifically from 1 to 6 carbon atoms. Specifically, examples of “C₁₋₆ alkyl group” include methyl group, ethyl group, n-propyl group, iso-propyl group, n-butyl group, iso-butyl group, sec-butyl group, tert-butyl group, n-pentyl group, 1,1-dimethylpropyl group, 1,2-dimethylpropyl group, 2,2-dimethylpropyl group, 1-ethylpropyl group, n-hexyl group, 1-ethyl-2-methylpropyl group, 1,1,2-trimethylpropyl group, 1-ethylbutyl group, 1-methylbutyl group, 2-methylbutyl group, 1,1-dimethylbutyl group, 1,2-dimethylbutyl group, 2,2-dimethylbutyl group, 1,3-dimethylbutyl group, 2,3-dimethylbutyl group, 2-ethylbutyl group, 2-methylpentyl group, 3-methylpentyl group and the like. A “C₁₋₄ alkyl group” is an alkyl group as defined above with 1, 2, 3 or 4 carbon atoms. Examples of “C₁₋₄ alkyl group” include methyl group, ethyl group, n-propyl group, iso-propyl group, n-butyl group, iso-butyl group, sec-butyl group and tert-butyl group. The alkyl group can be optionally interrupted by one or more oxygen atoms, preferably 1 to 4 oxygen atoms, more preferably 1 or 2 oxygen atoms.

The aryl group is preferably a “C₆₋₁₀ aryl group”, i.e. an aryl group constituted by 6, 7, 8, 9 or 10 carbon atoms. For the purposes of the invention, the aryl group includes condensed ring groups such as monocyclic ring group, or bicyclic ring group and the like. Specifically, examples of “C₆₋₄₀ aryl group” include phenyl group, indenyl group, naphthyl group or azulenyl group and the like. It should be noted that condensed rings such as indan and tetrahydro naphthalene are also included in the aryl group. The aryl group is optionally substituted with 1-4 substituent(s) selected from halogen, an oxo group, an ethylenedioxy group, methyl group, ethyl group, butyl group, methoxy group, methylamino group or dimethylamino group.

The arylalkyl group can be positioned such that the aryl or the alkyl group is the most remote from the molecule.

The alkoxy group is preferably a “C₁₋₆ alkyloxy group” meaning an oxy group that is bonded to an alkyl group (as previously defined). Specifically, examples of “C₁₋₆ alkoxy group” include methoxy group, ethoxy group, n-propoxy group, iso-propoxy group, n-butoxy group, iso-butoxy group, sec-butoxy group, tert-butoxy group, n-pentyloxy group, iso-pentyloxy group, sec-pentyloxy group, n-hexyloxy group, iso-hexyloxy group, 1,1-dimethylpropoxy group, 1, 2-1 dimethylpropoxy group, 2,2-dimethylpropoxy group, 2-methylbutoxy group, 1-ethyl-2-methylpropoxy group, 1,1,2-trimethylpropoxy group, 1,1-dimethylbutoxy group, 1,2-dimethylbutoxy group, 2,2-dimethylbutoxy group, 2,3-dimethylbutoxy group, 1,3-dimethylbutoxy group, 2-ethylbutoxy group, 2-methylpentyloxy group, 3-methylpentyloxy group and the like.

The arylaklyloxy group is an alkyloxy group as defined here, together with an attached aryl group. The arylalkyloxy group can be positioned so that the aryl group or the alkyloxy group is the most remote from the molecule.

It will be appreciated that the compounds of formula (I) are derivatives of the dipeptide Tyrosyl-Arginine. For the purpose of the present invention, the amino acid monomers tyrosine and arginine can independently be in the L or D configuration. The present invention therefore encompasses compounds of formula (I) comprising the backbone L-Tyrosyl-L-Arginine, L-Tyrosyl-D-Arginine, D-Tyrosyl-L-Arginine or D-Tyrosyl-D-Arginine. The compound of formula (I) may comprise L-Tyrosyl-L-Arginine.

The second aspect of the invention relates to the use of the compound of formula (I) in the manufacture of a systemic medicament for the prevention and/or treatment of pain.

The third aspect of the invention relates to a systemic pharmaceutical composition comprising the compound of formula (I) and a pharmaceutically acceptable excipient.

For the purposes of this invention, the pharmaceutically acceptable excipient may be a pharmaceutically acceptable carrier and/or a pharmaceutically acceptable diluent. Suitable carriers and/or diluents are well known in the art and include pharmaceutical grade starch, mannitol, lactose, magnesium stearate, sodium saccharin, talcum, cellulose, glucose, sucrose (or other sugar), magnesium carbonate, gelatin, oil, alcohol, detergents, emulsifiers or water (preferably sterile). The composition may be a mixed preparation of a composition or may be a combined preparation for simultaneous, separate or sequential use (including administration).

For formulation, a diluent, a binder, a disintegration agent, a lubricant, a colorant and a flavoring agent used in general, and as necessary, additives such as a stabilizer, an emulsifier, an absorption enhancer, a surfactant, a pH adjuster, an antiseptic agent, and an antioxidant can be used in the pharmaceutical composition. In addition, formulation is also possible by combining ingredients that are used in general as raw materials of pharmaceutical formulation, by the conventional method. Examples of these ingredients include (1) soybean oil, animal oil such as beef tallow and synthethic glyceride; (2) hydrocarbon such as liquid paraffin, squalane and solid paraffin; (3) an ester oil such as octyldodecylmyristate and isopropylmyristate; (4) higher alcohol such as cetostearylalcohol and behenyl alcohol; (5) a silicon resin; (6) a silicon oil; (7) a surfactant such as polyoxyethylene fatty acid ester, sorbitan fatty acid ester, glycerin fatty acid ester, polyoxyethylene sorbitan fatty acid ester, polyoxyethylene hardened castor oil and polyoxyethylene polyoxypropylene block co-polymer; (8) a water-soluble polymer such as hydroxyethyl cellulose, polyacrylic acid, carboxyvinyl polymer, polyethyleneglycol, polyvinylpyrrolidone and methyl cellulose; (9) lower alcohol such as ethanol and isopropanol; (10) multivalent alcohol such as glycerin, propylene glucol, dipropylene glycol and sorbitol; (11) a sugar such as glucose and cane sugar; (12) an inorganic powder such as anhydrous silicic acid, magnesium aluminium silicate and aluminium silicate; and (13) purified water and the like.

Among the aforementioned additives, use can be made of 1) lactose, corn starch, sucrose, glucose, mannitol, sorbit, crystalline cellulose, silicon dioxide and the like as a diluting agent; 2) polyvinyl alcohol, polyvinyl ether, methyl cellulose, ethyl cellulose, gum arabic, traganth, gelatine, shellac, hydroxypropyl cellulose, hydroxypropylmethyl cellulose, polyvinylpyrrolidone, polypropyleneglycol polyoxyethylene block co-polymer, meglumine, calcium citrate, dextrin, pectin and the like as a binder; 3) a starch, agar, gelatine powder, crystalline cellulose, calcium carbonate, sodium bicarbonate, calcium citrate, dextrin, pectin, calcium carboxymethylcellulose and the like as a disintegration agent; 4) magnesium stearate, talc, polyethyleneglycol, silica, hardened plant oil and the like as a lubricant; 5) a colorant, as long as addition thereof to a pharmaceutical drug is authorized, as a colorant; 6) a cocoa powder, menthol, fragrance, a peppermint oil, a cinnamon powder as a flavoring agent; and 7) an antioxidants whose addition to a pharmaceutical drug is authorized such as ascorbic acid and α-tocophenol as an antioxidant.

The fourth aspect of the invention relates to the systemic pharmaceutical composition of the third aspect for use in the prevention and/or treatment of pain.

The fifth aspect of the invention relates to a method of preventing and/or treating pain comprising the administration to a patient in need thereof of the compound of formula (I), wherein said the compound of formula (I) is administered systemically.

A sixth aspect of the invention relates to a compound selected from the group consisting of a compound of formula I, as defined in claim 1, wherein
X and Z are hydrogen, T is hydroxyl and Y is NH₂, in the form L-Tyrosyl-D-Arginine-NH₂, or D-Tyrosyl-D Arginine-NH₂ or D-Tyrosyl-L-Arginine-NH₂; or
X is Ibuprofen, Z is hydrogen, T is hydroxyl and Y is NH₂ in the form Ibuprofen-L-Tyrosyl-L-Arginine-NH₂ or Ibuprofen-D-Tyrosyl-L-Arginine-NH₂, or
X is Ibuprofen, Z is hydrogen and T and Y are hydroxyl in the form Ibuprofen-L-Tyrosyl-L-Arginine-OH, or
X is methyl, Z is hydrogen, T is hydroxyl and Y is NH₂, in the form methyl-L-Tyrosyl-L-Arginine-NH₂ or methyl-L-Tyrosyl-D-Arginine-NH₂, optionally in the form of a pharmaceutical composition.
   Such a compound in the form of a pharmaceutical formulation involves the compound and a pharmaceutically acceptable excipient as herein described.

The compound of formula (I) according to the invention for use in the aforementioned indications may be administered by any convenient method, for example by enteral or parenteral administration as hereinbefore described and the composition adapted accordingly.

The compound of formula (I) according to the present invention can be provided in a delayed release composition in combination with a delayed release component to allow targeted release of the compound of formula (I) into the lower gastrointestinal tract for example into the small intestine, the large intestine, the colon and/or the rectum. The delayed release component may comprise an enteric or pH dependent coating, hydrophobic or gelling excipients or coatings, by time dependent hydrogel coatings and/or by acrylic acid linked to azoaromatic bonds coatings.

For oral administration, the compound can be formulated as liquids or solids, for example solutions, syrups, suspensions, emulsions, tablets, capsules, lozenges, dry powder and/or granules.

A liquid formulation will generally consist of a suspension or solution of the compound or physiologically acceptable salt in a suitable aqueous or non-aqueous liquid carrier(s) for example water, ethanol, glycerol, polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and microcrystalline cellulose.

A composition in the form of a capsule can be prepared using routine encapsulation procedures. For example, powders, granules or pellets containing the active ingredient can be prepared using standard carriers and then filled into a capsule, for example a hard gelatin capsule, a HPMC capsule, a soft gelatin capsule etc; alternatively, a dispersion or suspension can be prepared using any suitable pharmaceutical carrier(s), for example aqueous gums, celluloses, silicates or oils and the dispersion or suspension then filled into a soft gelatin capsule.

Compositions for oral administration may be designed to protect the active ingredient against degradation as it passes through the alimentary tract, for example by an outer coating of the formulation on a tablet or capsule.

Typical parenteral compositions consist of a solution or suspension of the compound or physiologically acceptable salt in a sterile aqueous carrier or non-aqueous or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal or oral administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a pharmaceutically acceptable propellant. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal or vaginal administration are conveniently in the form of suppositories (containing a conventional suppository base such as cocoa butter), pessaries, vaginal tabs, foams or enemas.

Conveniently the composition is in unit dose form such as a tablet, capsule or ampoule.

The composition may contain from 0.1% to 99% (w/w) preferably from 0.1-60% (w/w), more preferably 0.2-20% by weight and most preferably 0.25 to 12% (w/w) of the compound of formula (I), depending on the method of administration.

The compound of formula (I) is provided for the prevention and/or treatment of pain in a human or an animal. The compounds of the invention are therefore provided for both medical and veterinary use. References in the application to the administration of the compound of formula (I) to “a patient” therefore include administration to a human and/or to an animal, more specifically to a mammal. The compound of formula (I) is preferably provided for administration to a human. For the purposes of this invention, the compound of formula (I) is also particularly provided for the prevention and/or treatment of pain in companion animals (such as a cat, dog, rodent, horse etc), farm animals (such as poultry, a sheep, a cow, a pig) or animals in captivity (such as zoo animals).

The amount of the compound of formula (I) effective to treat pain depends on the nature and severity of the pain being treated and the weight of the patient in need thereof. The compounds of the invention will normally be administered in a daily dosage regimen (for an adult human patient) of, for example, an oral dose of between 1 mg and 2000 mg, preferably between 30 mg and 1000 mg, e.g. between 10 and 250 mg or an intravenous, subcutaneous, or intramuscular dose of between 0.1 mg and 100 mg, preferably between 0.1 mg and 50 mg, e.g. between 1 and 25 mg of the compound of the formula (I) or a physiologically acceptable salt thereof calculated as the free base, the compound being administered 1 to 4 times per day. The unit dose is preferably provided in the form of a capsule or a tablet. Suitably the compounds will be administered for a period of continuous therapy, for example for a week or more. It will be appreciated that the dose ranges set out above provided guidance for the administration of the compound of formula (I) to an adult. The amount to be administered to for example, an infant or a baby can be determined by a medical practioner or person skilled in the art and can be lower or the same as that administered to an adult.

All preferred features of each of the aspects of the invention apply to all other aspects mutatis mutandis.

It is noted here that in this text, unless otherwise specified, KTP-NH₂ is the compound of the present invention where X is hydrogen, Z is hydrogen, T is hydroxyl and Y is NH₂ in the L-L form.

The invention may be put into practice in various ways and a number of specific embodiments will be described by way of example to illustrate the invention with reference to the accompanying drawings, in which:

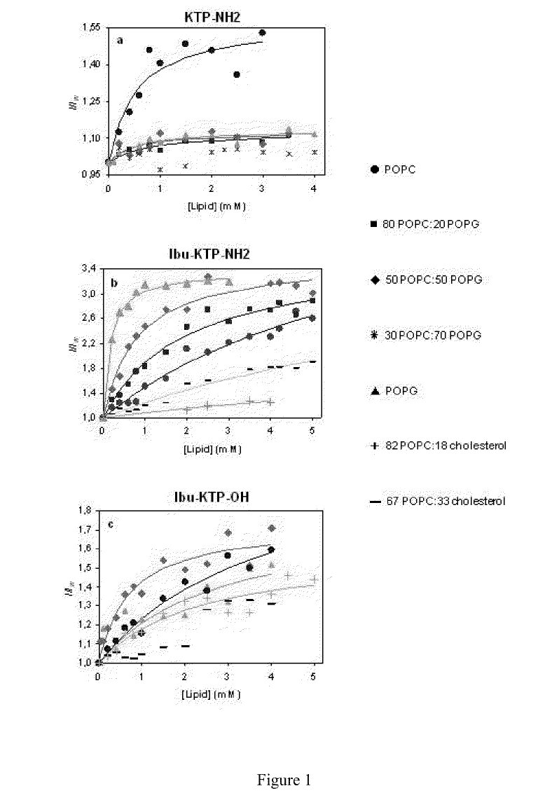

FIG. 1 shows a partition curve for (a) KTP-NH2 (X and Z are hydrogen, T is OH and Y is NH₂), (b) Ibu-KTP-NH2 (X is ibuprofen, Z is hydrogen, T is OH and Y is NH₂) and (c) Ibu-KTP (X is ibuprofen, Z is hydrogen, T is OH and Y is OH). Vesicles of zwiterionic lipid, POPC (), and POPC with negative lipid, POPG in different proportions: 80:20 (▪), 50:50 (♦) and 30:70 (*) until 100% POPG). Vesicles made by lipid mixtures of POPC and cholesterol with proportions of 82:18 (−) and 67:33 (+) are also shown for Ibu-KTP-NH2 and Ibu-KTP.

The unit of l/lw on the y axis is the ratio between fluorescence intensities.

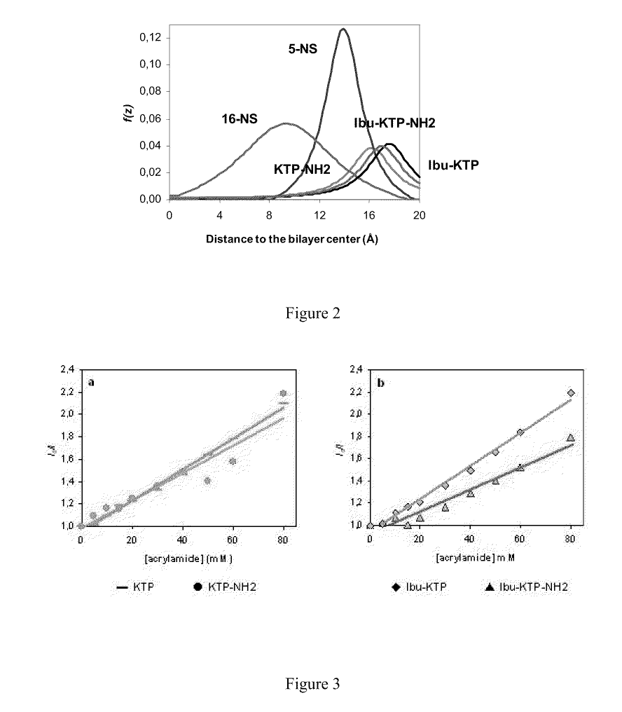

FIG. 2 shows peptide distribution for a representative lipidic system—KTP-NH₂ (furthest left of test results), Ibu-KTP-NH₂ (middle line of test results) and Ibu-KTP (furthest right of test results). The x-axis indicates the distance to the bilayer center, being the monolayer thickness 20 Å and y-axis represents the values of the probability density function. Lines noted as 16-NS and 5-NS relate to the location of the quenching agents used in the study;

FIG. 3 shows a Stern-Volmer plot for fluorescence quenching of kyotorphins: (a) KTP (X and Z are hydrogen, T and Y are OH) (−), KTP-NH₂ and (b) Ibu-KTP (♦) Ibu-KTP-NH₂ (▴);

The unit of lo/l on the y axis is the ratio between fluorescence intensities.

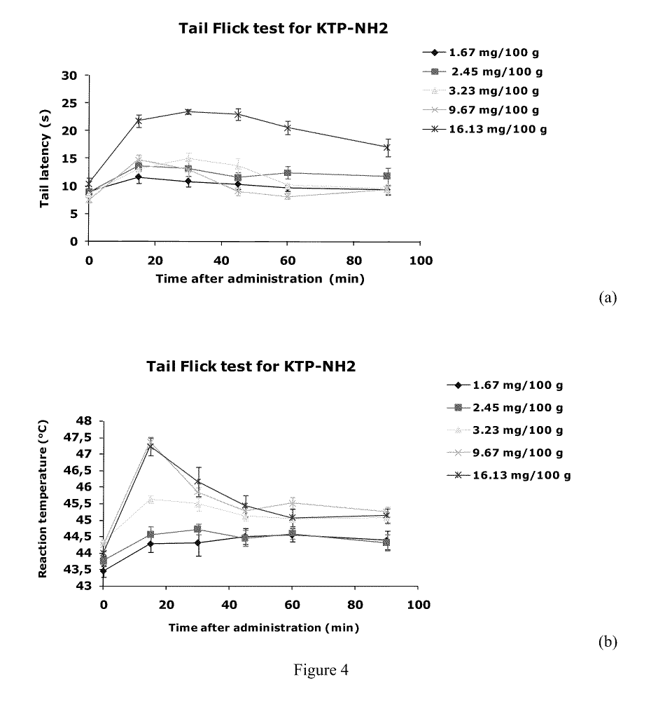

FIG. 4 shows dose-response curves for KTP-NH₂ i.p., for (a) Tail Flick and (b) Hot Plate tests;

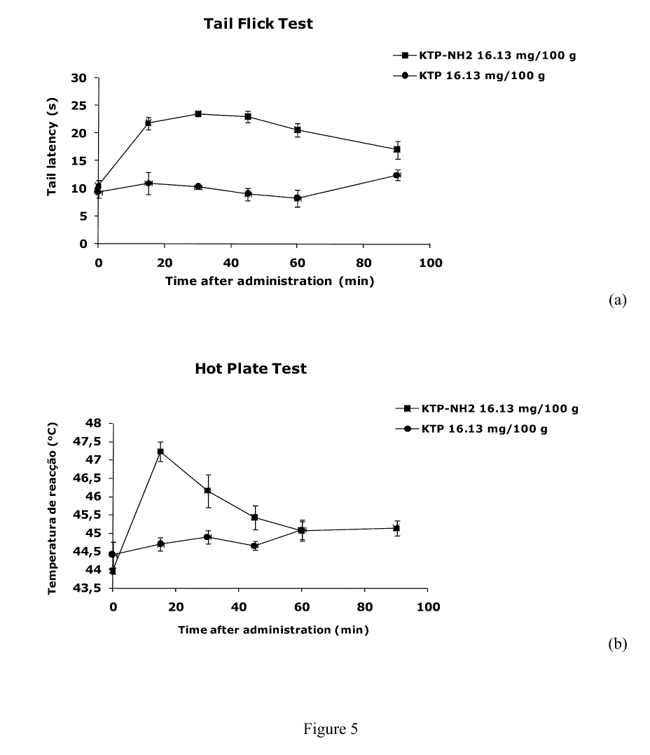

FIG. 5 shows a comparison between analgesic efficacy in KTP-NH₂ and KTP produced by i.p. administration for (a) Tail Flick and (b) Hot Plate tests;

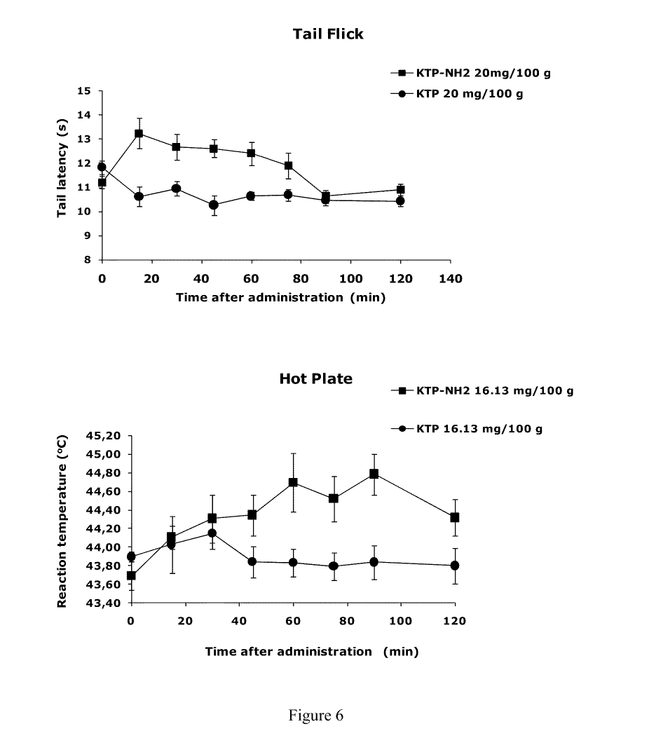

FIG. 6 shows dose-response curves for oral administration for Tail Flick and Hot Plate tests

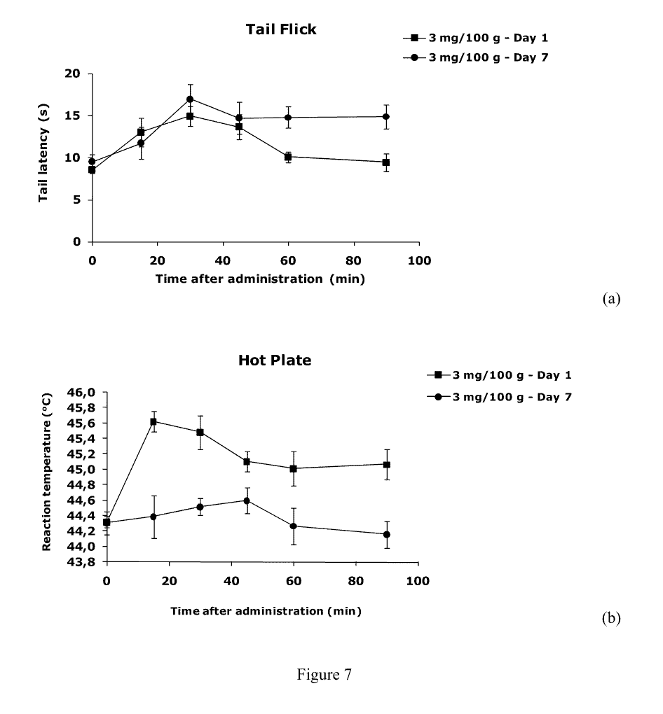

FIG. 7 shows (a) Tail Flick and (b) Hot Plate tests for rats injected with a dose of 3.23 mg/100 g of body mass during 7 days (diamonds) compared to positive control—rats injected once (squares);

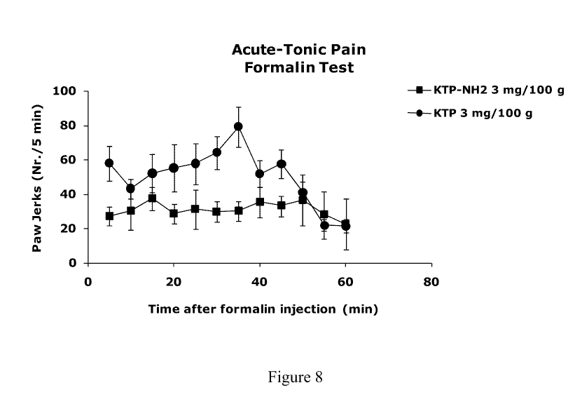

FIG. 8 shows formalin test results for acute-tonic pain therapeutic assessment of KTP-NH₂;

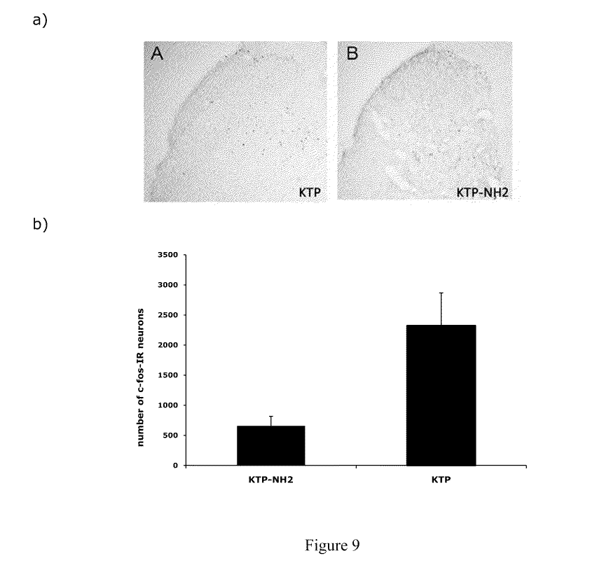

FIG. 9 shows that KTP-NH₂ treatment reduced the number of immunoreactive c-fos neurons in the dorsal horn of formalin-treated rats: a) histological sections of the dorsal horn; b) quantification of immunoreactive neurons in KTP-NH₂ treated and control rats;

FIG. 10 shows an analgesic effect of KTP-NH₂ in comparison with KTP in monoarthritis rats for (a) Hargreaves and (b) Tail Flick tests;

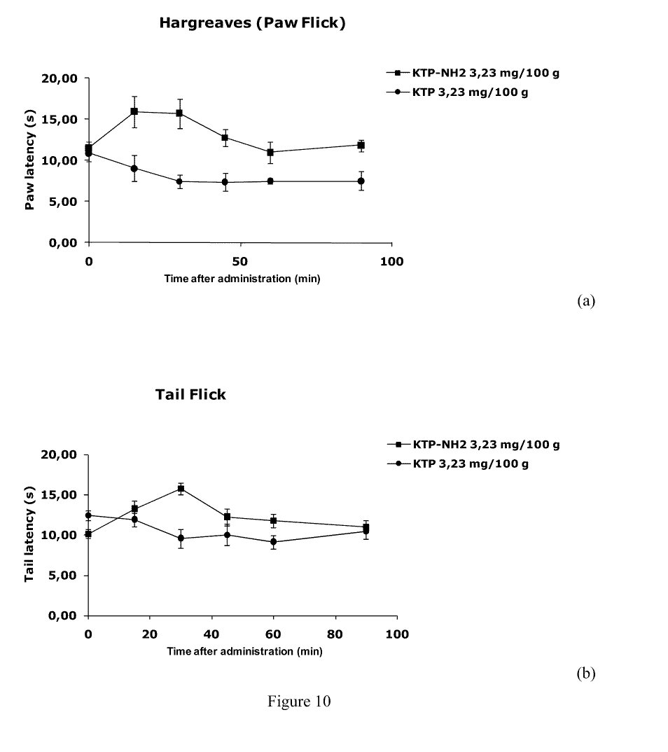

FIG. 11 shows a comparison of KTP-NH₂ and ibuprofen analgesic efficacy. Compounds were administered i.p.;

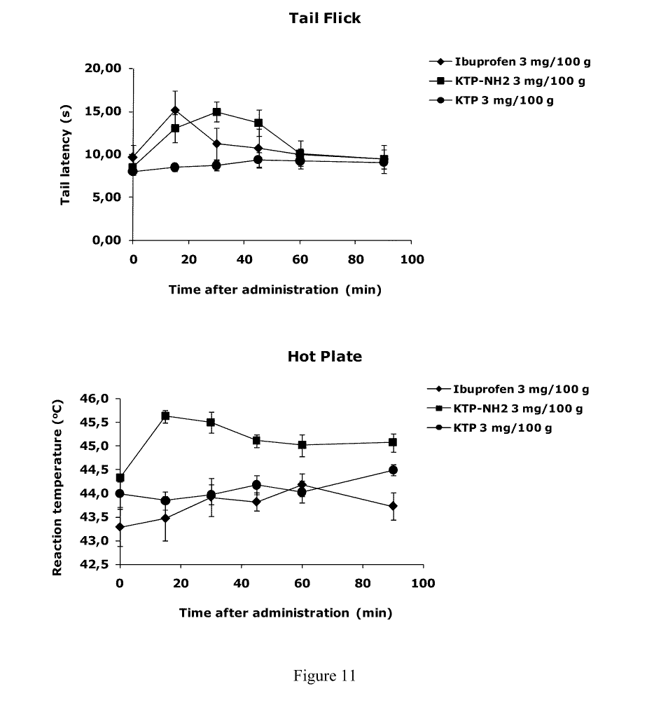

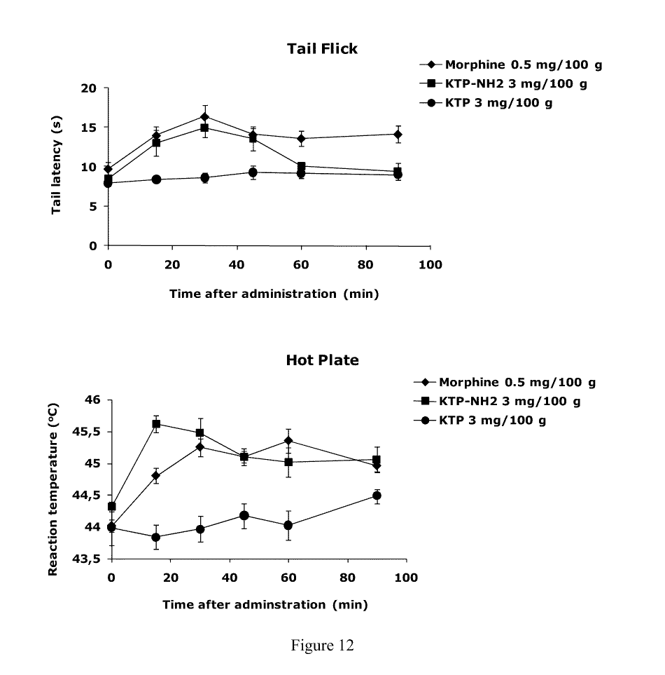

FIG. 12 shows a comparison of KTP-NH₂ and morphine analgesic efficacy. Compounds were administered i.p.;

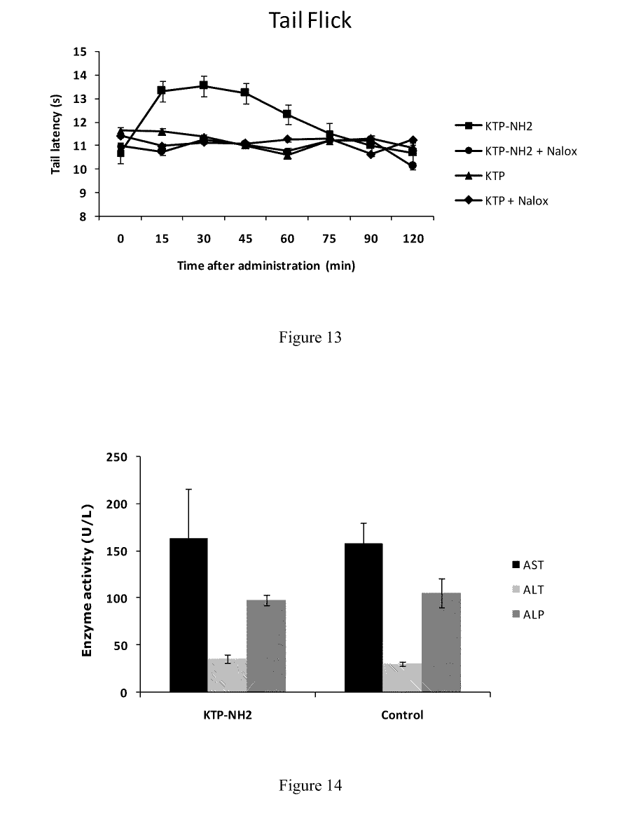

FIG. 13 shows the effects of naxolone on rat tail-flick response. KTP-NH₂ and KTP were administered at 3 mg/100 g, naxolone was administered at 0.5 mg/100 g;

FIG. 14 shows the enzyme activity (U/L) of AST, ALT and ALP measured in the plasma of rats treated with a daily dose of 3.23 mg/100 g of body mass during 7 days compared with control animals;

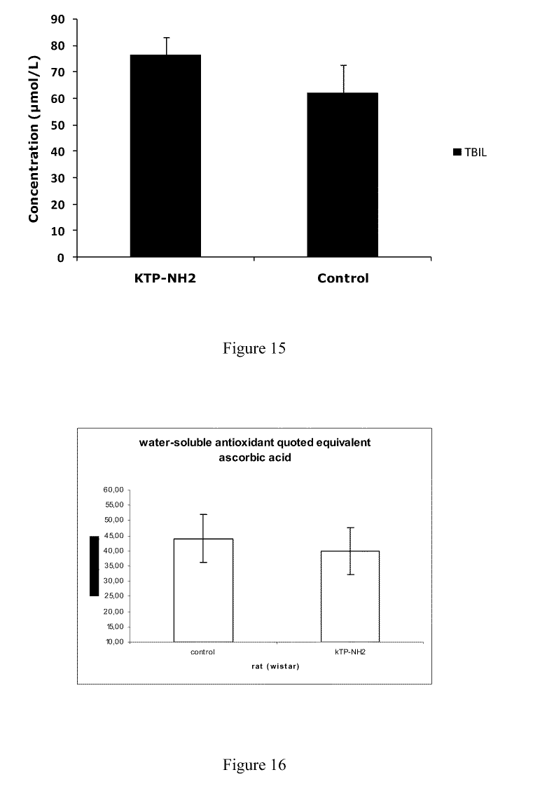

FIG. 15 shows the total bilirubin quantity (μmol/L) measured in plasma of rats treated with a daily dose of 3.23 mg/100 g of body mass during 7 days compared with control animals;

FIG. 16 shows the antioxidant capacity of water-soluble constituents in plasma of treated rats. The values are quoted in equivalent ascorbic acid (μmol/L); and

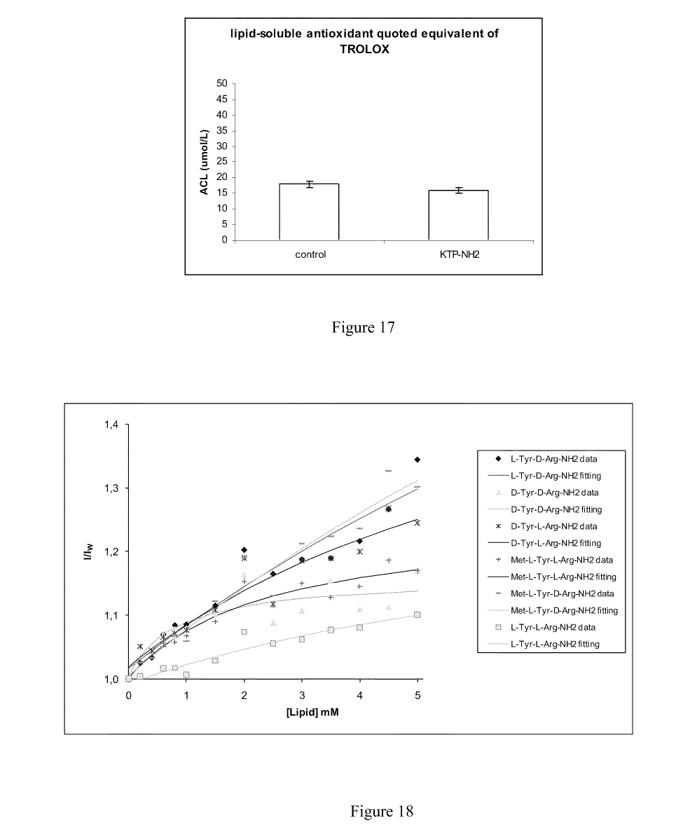

FIG. 17 shows the antioxidant capacity of lipid-soluble constituents in plasma of treated rats. The values are quoted in equivalent TROLOX (6-Hydroxy-2,5,7,8-tetramethylchromane-2-carboxylic acid) (μmol/L).

FIG. 18 shows partition curves for derivatives of KTP-NH2 with improved plasma stability. Vesicles of zwiterionic lipid, POPC, and POPC with negative lipid, POPG, in the proportion 50 POPC: 50 POPG;

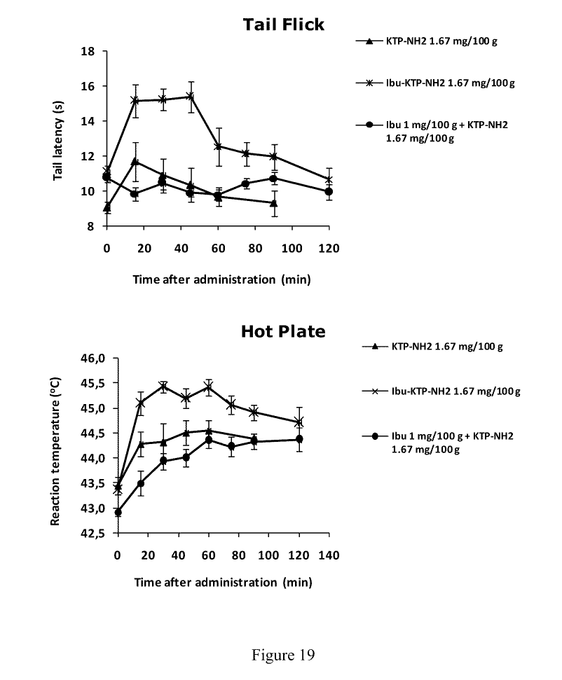

FIG. 19 shows the increased analgesic efficacy of Ibu-KTP-NH₂ produced by i.p. administration for (a) Tail Flick and (b) Hot Plate tests.

### EXAMPLES

### Biophysical Studies

### Partition Curves of KTP-NH₂; KTP-NH₂-Derv and KTP-Derv

We performed biophysical studies using fluorescent methodologies in order to characterize the interaction of KTP-NH2 with liposomes representing human cell membranes (Santos N. C., Prieto M. and Castanho M. A. 2003. Quantifying molecular partition into model systems of biomembranes: an emphasis on optical spectroscopic methods. Biochim Biophys Acta 1612: 123-135.). A clear interaction of KTP-NH2 with the lipid bilayers was observed. FIG. 1 demonstrates the titration of three derivatives of KTP, (a) KTP-NH₂ (X and Z are hydrogen, T is OH and Y is NH₂), (b) Ibu-KTP-NH2 (X is ibuprofen, Z is hydrogen, T is OH and Y is NH₂) and (c) Ibu-KTP (X is ibuprofen, Z is hydrogen, T is OH and Y is OH) with a mammal-mimetic lipid bilayer vesicle. When titrated with a mammal-mimetic lipid bilayer vesicle (circles), the fluorescence intensity of the phenolic moiety increases due to insertion in the apolar environment created by the lipids. The POPC system mimetizes the external layer of human cell membranes, whereas POPG lipidic mixture mimetizes the internal layer of human cell membranes. Numerical treatment of the data shows that the local concentration in the model membrane of mammals is approx. 2500; 190 and 333 fold higher than in the surrounding bulk aqueous phase, for KTP-NH₂, Ibu-KTP-NH₂ and Ibu-KTP, respectively. These results show that KTP-NH2 is 2500 fold more concentrated in the lipid bilayers than in the surrounding non-lipidic moiety, indicating a high liposolubility.

### Depth Distribution of the Peptides in the Membrane

Differential quenching experiments were used to estimate the depth of location of the phenolic ring of the peptides, based on a Brownian dynamics simulation (Fernandes M. X., Garcia de la Torre J., Castanho M. A. 2002. Joint determination by Brownian dynamics and fluorescence quenching of the in-depth location profile of biomolecules in membranes. Anal. Biochem. 307: 1-12.) This methodology allows to determine where the molecules are preferably inserted along the lipid bilayer. The results are shown in FIG. 2. The peptides with the amide group show a greater depth of insertion, 4 Å—KTP-NH₂ and 3 Å—Ibu-KTP-NH₂, from the lipid/water interface. This is in agreement with KTP-NH2 having a higher affinity for lipids, as desired.

### Acrylamide Quenching Studies

The aggregation of molecules in aqueous medium, as a result of poor solubility, for example, is a critical parameter in pharmacology. As we generated a molecule with increased lipophilicity, KTP-NH2, it was crucial to demonstrate that this molecule and derivatives thereof maintained appropriate solubility characteristics. For this purpose, acrylamide quenching studies were performed (Lakowicz J. R 1999. Principles of Fluorescence spectroscopy, Second Edition ed., Kluwer Academic/Plenum Publishers, New York; Coutinho A and Prieto M. 1993. Ribonuclease T1 and alcohol dehydrogenase fluorescence quenching by acrylamide: A laboratory experiment for undergraduate students J. Chem. Education, 70: 425-428). Acrylamide is an aqueous quencher of tyrosine fluorescence. When fluorescent peptides aggregate, the phenolic groups tend to cluster and remain inaccessible to hydrophilic molecules. Conversely, when no aggregation occurs, all tyrosine residues are exposed to the solvent and, therefore, accessible to the contact and quenching by acrilamide. The linearity observed in FIG. 3 a) and b) show that the phenolic groups are always accessible to the hydrophilic molecule acrylamide. The fluorescence spectra were not concentration-dependent (not shown) and there was a linear dependence of fluorescence intensity on concentration (not shown). These results show that no evidence for aggregation of these peptides in aqueous medium was found, indicating favourable solubility properties for KTP-NH2 and tested derivatives.

### Behaviour Results

In vivo behavioural anti-nociception tests were carried out in rats (Wister, male). Unless otherwise stated, compounds were administered by intra-peritoneal (i.p.) injection. For most assays, the Tail Flick and the Hot Plate tests were used.

The tail-flick test (D'Amour F. E., Smith D. L. 1941. A method for determining the loss of pain sensation. J Pharmacol Exp Ther 1941; 72:74-79) is a standard investigative tool for pain and analgesia assessment in rodents. It is based on a spinal reflex response of the tail to radiant heat. Pain sensitivity in rats was measured as they responded to the application of radiant heat to a small area of their tails. The rat's tail was placed over a window located on a platform and subjected to irradiation by an intense light beam (10 W). When the rat feels discomfort, it flicks its tail which automatically stops the timer. The reaction time from activation of the light beam to the tail flick is automatically presented on a digital display. A cut-off time of 24 s was applied. Animal reaction time is a measurement of animal resistance to pain and is used to measure efficacy of analgesics.

The hot-plate test (Eddy N. B and Leimbach D. 1953. Synthetic analgesics II. Dithienylbutenyl and dithienylbutylamines J. Pharmacol. Exp. Ther. 45: 339) evaluates a supraspinally integrated response in the form of thermal pain reflexes due to footpad contact with a heated surface. During the experiments, the animal is confined in a removable clear acrylic compartment where the latency time to the first hind paw or/and jumping responses are measured. We used a modified hot plate test (Hunskaar S., Berge O. G., Hole K. 1986. A modified hot-plate test sensitive to mild analgesics. Behay. Brain Res. 21: 101-108) in which the temperature was slowly increased (9° C./min) from non-noxious levels (35° C.) until a response was observed or a cut-off temperature was reached (52.5° C.). The response is the licking of the hindpaw, and the corresponding plate temperature represents the recorded nociceptive end-point. The advantage of Increasing-Temperature Hot-Plate test over the standard constant-temperature hot-plate test is the higher sensitivity and the lack of influences of pre-exposure to the hot-plate before testing. Animal reaction temperature is a measurement of animal resistance to pain and is used to measure efficacy of analgesics.

### Acute Pain Models: Tail Flick and Hot Plate Tests

Dose-response curves (FIG. 4) demonstrate a correlation trend between analgesic effect and dose. It should also be stressed that the smallest dose tested with statistical significance was 1.67 mg and 2.45 mg for 100 g of body mass, for Hot Plate and Tail Flick tests, respectively.

Even high doses of KTP (X and Z are hydrogen, T and Y are OH) show a very small effect on the tail flick latency and the hot plate test in the rat (FIG. 5). In contrast, KTP-NH₂ demonstrates a clear anti-nociceptive response, which lasts for 45 min in Tail Flick and 30 min in Hot Plate (also FIG. 5).

### Acute Pain Models: Tail Flick and Hot Plate Tests (Oral Administration)

In order to assess the potential for oral administration of KTP-NH2, the analgesic efficacy of KTP-NH2 was tested by in vivo behavioral nociception tests (Tail Flick and Hot Plate) after oral administration of the compound in rats (Wister, male) (FIG. 6). We observed a slight delay of the onset of effect, as expected for an oral administration profile. These results prove the potential of KTP-NH2 to be administrated as an oral drug, highly improving its pharmacological value.

### Chronic Administration of Peptide—Tail Flick and Hot Plate Tests

In order to make a first evaluation of the toxicology of the peptide, rats were injected once a day with 3.23 mg/100 g body mass for seven days. On the last day, the rats were tested to see if the peptide was still able to produce and effect. The results are shown in FIG. 7.

“Chronic” administration does not impair the rats for being respondent. In the Tail Flick test, which is related to a spinal reflex, rats were still tolerant to radiation, in a very similar profile to the control (of a single injection). This evidences good results even with continual administration of the peptide.

### Inflammatory Pain Model: Formalin Test

The formalin test was introduced as a model of tonic pain in 1977 (Dubuisson D., Dennis S. G. 1977. The formalin test: a quantitative study of the analgesic effect of morphine, meperidine and brain stem stimulation in rats and cats. Pain; 4:74-161). In rats, formalin generates an initial phase of activity (phase 1, acute), a quiescent interphase, and a second phase of activity (phase 2, tonic/chronic), and this is seen with spontaneous behaviors, firing of afferent neurons, and activity in dorsal horn neurons. Both active phases involve ongoing peripheral afferent neural activity; inflammation contributes to phase 2 activity and the interphase results from active inhibition. Within the spinal cord, formalin increases c-fos expression in neurons and causes activation of microglia, and these may contribute more prominently to longer term changes.

We performed the formalin test due to its validity as a model to study central sensitization events at the spinal level after peripheral inflammatory states. The standard two types of nociception were analyzed; the short-lasting nociception caused by a direct effect on nociceptors followed by a long-lasting nociception due to inflammation. KTP-NH2 was administered i.p. 10 min previous to formalin injection. Rats were treated with 5% neutral formalin s.c. (sub-cutaneous) injection in the hind paw to induce acute-tonic pain. The paw jerks were videotaped and measured both in the acute and in the tonic-chronic phases. KTP-NH2 reduced significantly the number of paw-jerks in both pain phases (FIG. 8). Painful stimulation produced by formalin induces c-fos expression in the spinal cord and c-fos is considered the most relevant molecular marker for pain. The effects of KTP-NH2 on the formalin-induced c-fos expression in the spinal cord dorsal horn were examined Immunohistochemistry against c-fos confirmed that, consistently with the behavioral results observed, KTP-NH2 significantly decreased formalin-induced c-fos protein expression in dorsal horn, validating its therapeutic potential in the treatment of inflammatory pain (FIG. 9).

### Chronic Inflammatory Pain Model—Monoarthritis (CFA-Induced)—Tail Flick and Hargreaves (Paw Flick) Tests

The subcutaneous intraplantar injection of complete Freund's adjuvant (CFA) into the local area of an animal can cause severe inflammatory pain around the injected area. This CFA-induced inflammatory pain has been widely used as a kind of pain model in the field of pain research since it was reported. Inflammatory pain was induced by an injection of complete Freund's adjuvant into the right hindpaw of the rats (S. H. Butler, F. Godefroy, J. M. Besson and J. Weilfugazza. 1992. A limited arthritic model for chronic pain studies in the rat. Pain, 48: 73-81). The paw withdrawal latency was used to assess the inflammatory pain according to the method of Hargreaves et al. (Hargreaves K, Dubner R, Brown F, Flores C, and Joris J. 1988. A new and sensitive method for measuring thermal nociception in cutaneous hyperalgesia. Pain 32: 77-88, 1988). The CFA-injected area of the right paw was placed on a constant-intensity radiant heat source and the time of paw-withdrawal latency was measured by observing the animal's paw-withdrawal response after applying initial heat to the left or right paws. Additionally, the Tail Flick test was performed in order to assess the secondary hyperalgesia.

FIG. 10, shows the efficacy of KTP-NH₂ in the treatment of chronic inflammatory pain. These results suggest that this compound is a good candidate both for primary and secondary hyperalgesia.

Analgesic Potential—Comparison with Marketed Analgesic: Ibuprofen and Morphine

The analgesic potential of KTP-NH2 was also verified by comparison with well-known, widely-used analgesic molecules: ibuprofen and morphine. In vivo behavioral nociception tests revealed that KTP-NH2 performs better than ibuprofen, showing a more pronounced effect in the Hot Plate test and a more durable effect in the Tail Flick test (FIG. 11). Regardless of the fact that both molecules may be using different analgesic mechanisms, these results show that KTP-NH2 is an effective analgesic compound.

From FIG. 12 it is possible to observe that the efficacy of 3 mg/100 g of KTP-NH2 is similar to that observed with 0.5 mg/100 g dose of morphine. However, rats injected with morphine revealed several alterations in behavior, like scratching (common to patients on morphine treatment), whereas with KTP-NH2 no such secondary effects were observed, even at higher doses.

### Central Action Confirmation—Naloxone-Reversible Analgesia

Naloxone is a drug with high affinity for μ-opioid receptors in the central nervous system. Naloxone also has an antagonist action, though with a lower affinity, at κ- and δ-opioid receptors. In order to corroborate the evidence that KTP-NH2 was exerting its analgesic effect via a central action, we analyzed the antinociceptive effect of KTP-NH2 after pretreatment with naloxone. Administration of naloxone 10 min before administration of KTP-NH2 completely antagonized the analgesia (FIG. 13). These results indicate that KTP-NH2 analgesia is mediated via a central mechanism.

### Secondary Effects: Motor Ability Evaluation—Rota Rod Test

To evaluate the motor ability of animals after being injected with the drug (3.23 mg/100 g of body mass), the Rota-rod test was used. Briefly, the animal is placed on a wheel and run in balanced position; if balance is lost, the animal falls.

No loss of balance/equilibrium was noticed. The analgesic effect is therefore present without seeing any drowsy state.

### In Vivo Toxicology

The toxicological studies were carried out with rats (Wister, male) injected i.p. once a day with 3.23 mg/100 g body mass during seven days.

### Determination of Hepatotoxicity Markers in Plasma

Liver is the major organ of toxicity. Blood samples were collected and the following hepatotoxicity markers were assayed in the plasma:
Alanine transaminase (ALT),
Aspartate transaminase (AST)
Alkaline phosphatase (ALP)
Total bilirubin (TBILI)
Gamma glutamyl transpeptidase (GGT)

Increased levels of the liver enzymes ALT, AST, ALP and GGT in the plasma would indicate lesions in liver. Increased RBL would be a sign of faulty bilirubin production/hemolysis and/or bilirubin metabolism (in liver).

The results for levels of AST, ALT and ALP are shown in FIG. 14. The results for the level of TBILI are shown in FIG. 15.

Control and KTP-NH₂-treated animals show identical results: no toxic effects were detected.

Furthermore, in the test animals GGT was only present in trace amounts, below the detection limit of the analytical spectrophotometers.

### Determination of Antioxidant Capacity in Blood Samples

Metabolization of KTP-NH2 in the organism might lead to an increase in its metabolite products and to the subsequent production of reactive oxygen species (ROS). As increased ROS levels are potentially harmful, KTP-NH₂ potential to induce changes in antioxidant capacities in the plasma of animals was explored. Total antioxidant capacity of lipid-soluble (ACL) and water-soluble (ACW) constituents were determined. As set out in FIGS. 16 and 17, the drug does not induce significant modifications of basal antioxidant capacity in plasma of treated animals.

### Detection of Histological Alterations

For investigation of histological alterations in liver, kidney and spleen hematoxylin/eosin-stained sections of fixed tissue were used. Hematoxylin/eosin-stained sections were examined by an experienced pathologist There were no sign of hepatic necrosis in rat's liver and no histological modifications were found in kidney and spleen of KTP-NH2-treated animals.

### In Vitro ADMET (Absorption, Distribution, Metabolism, Excretion and Toxicology).

ADMET deficient properties are one of the major factors that cause failures during drug development. Therefore, the ADME characteristics of KTP-NH2 were evaluated in vitro and the compound proved to have attributes of a good drug candidate.

### Metabolic Stability

Many compounds can never become a drug because they are rapidly metabolized in the liver. It is important to confirm that metabolic stability is adequate to the desired distribution of compound throughout the body. The in vitro metabolic stability of KTP-NH2 was tested using a microsomal preparation from human liver, which contains all the cytochrome P450 (CYP) isozymes and other metabolizing enzymes (Kuhnz W. and Gieschen H. 1998. Predicting the oral bioavailability of 19-nortestosterone progestins in vivo from their metabolic stability in human liver microsomal preparations in vitro. Drug Metab. Dispos. 26: 1120-1127). Results obtained showed that KTP-NH2 is metabolically stable, as 93% of the compound remained after 1-hour incubation with the microsomal preparation.

### CYP Inhibition

If a compound inhibits cytochrome P450 (CYP) isozymes this will lead to the accumulation of endogenous substances or other drugs that are substrates of the inhibited CYP, leading to potential toxicity. CYP3A4 is one of the most important enzymes involved in the metabolism of xenobiotics in the body, promoting the oxidation of the largest range of substrates of all the CYPs and is present in the largest quantity of all the CYPs in the liver. KTP-NH2 was shown not to inhibit CYP3A4 in a specific in vitro inhibition assay (Dierks E. A., Stams K. R:, Lim H. K., Cornelius G., Zhang H. and Ball S. E. 2001. A method for the simultaneous evaluation of the activities of seven major human drug-metabolizing cytochrome P450s using an in vitro cocktail of probe substrates and fast gradient liquid chromatography tandem mass spectrometry. Drug Metab. Dispos. 29: 23-29) using two traditional CYP3A4 probe substrates, midazolan and testosterone.

### Cytotoxicity

Cytotoxicity was assessed with a cell-based assay using human hepatocytes. Cell death was assayed by quantifying plasma membrane damage or rupture through measurement of the release of lactate dehydrogenase (LDH) (Legrand, C. et al. 1992. Lactate dehydrogenase (LDH) activity of the cultured eukaryotic cells as marker of the number of dead cells in the medium. J. Biotechnol. 25: 231-243), a stable cytoplasmic enzyme present in most cells. Determination of cell viability was performed by quantifying ATP (Cree I. A and Andreotti P. E. 1997. Measurement of cytotoxicity by ATP-based luminescence assay in primary cell cultures and cell lines. Toxicology in vitro, 11: 553-556), a marker for cell viability because it is present in all metabolically active cells and the concentration declines very rapidly when the cells undergo necrosis or apoptosis. Treatment of human hepatocytes with increasing concentrations of KTP-NH2 and subsequent LDH and ATP determination revealed that the compound is low hazardous (LC50>125 μM).

### Plasma Stability

Drugs are exposed in plasma to enzymatic processes (proteinases, esterases), they can undergo intramolecular rearrangement or bind irreversibly (covalently) to proteins. Compounds which are not stable in plasma have inherent liability as drug candidates, as they are less capable to reach a sufficient concentration at their site of pharmacological activity. KTP-NH2 shows 14-21% stability in human plasma after 1 h incubation (Singh R., Chang S. Y. and Talor L. C. 1996. In vitro metabolism of a potent HIV-protease inhibitor (141 W94) using rat, monkey and human liver S9. Rapid Commun. Mass Spectrom. 10: 1019-1026), being metabolized into its constituent amino acids, arginine and tyrosine.

### Improved KTP-NH2 Analogues

In order to increase the potential of KTP-NH2 as a drug for systemic administration, derivatives with improved plasma stability were generated, and tested, including different isomers of KTP-NH2 and methylated versions of KTP-NH2 isomers:

L-Tyrosyl-D-Arginine-NH2

D-Tyrosyl-D-Arginine-NH2

D-Tyrosyl-L-Arginine-NH2

Ibuprofen-L-Tyrosyl-L-Arginine-NH₂

Ibuprofen-D-Tyrosyl-L-Arginine-NH₂,

Methyl-L-Tyrosyl-L-Arginine-NH2

Methyl-L-Tyrosyl-D-Arginine-NH2

These analogues were tested for plasma serum stability, in comparison with KTP-NH2. All of these KTP-NH2 analogues revealed reduced degradation and displayed high stability values, ranging from 51% to 99%, after 1-hour incubation, see table below.

| | Test | | Mean Parent |
|---|---|---|---|
| Compound | concentration [uM] | Condition | Remaining (%) |
| L-Tyr-L-Arg-NH2 | 400 | Human Serum | 14.00 |
| L-Tyr-D-Arg-NH2 | 400 | Human Serum | 78.78 |
| D-Tyr-D-Arg-NH2 | 400 | Human Serum | 98.37 |
| D-Tyr-L-Arg-NH2 | 400 | Human Serum | 98.01 |
| Me-L-Tyr-L-Arg-NH2 | 400 | Human Serum | 88.98 |
| Me-L-Tyr-D-Arg-NH2 | 400 | Human Serum | 98.47 |
| Ibu-L-Tyr-L-Arg-NH2 | 800 | Human Serum | 51.35 |
| Ibu-D-Tyr-L-Arg-NH2 | 800 | Human Serum | 94.08 |

In order to confirm that these plasma-stable KTP-NH2 derivatives maintained their lipophilicity, their potential interaction with human cell membranes was assessed. Again, we performed biophysical studies using fluorescent methodologies (Santos N. C., Prieto M. and Castanho M. A. 2003. Quantifying molecular partition into model systems of biomembranes: an emphasis on optical spectroscopic methods. Biochim Biophys Acta 1612: 123-135.). A good interaction of all of the improved derivatives with the lipid bilayers was corroborated by the results shown in FIG. 19, suggesting that they maintain the high lipophilic characteristics.

Finally, the analgesic efficacy of a plasma-stable derivative was assessed in vivo. FIG. 19 shows the improved effect of Ibu-KTP-NH2 over KTP-NH2, as observed for both the Hot Plate and the Tail Flick tests. These results corroborate the rational underlying the development of KTP derivatives: improved stability combined with blood brain barrier-permeation potential results in potent analgesic molecules.

### Synthesis of Compounds of the Invention

### Synthesis of Tyr-Arg-NH₂(KTP-NH2)

### Synthesis of Boc-Tyr(tBu)-Arg-NH₂ (3)

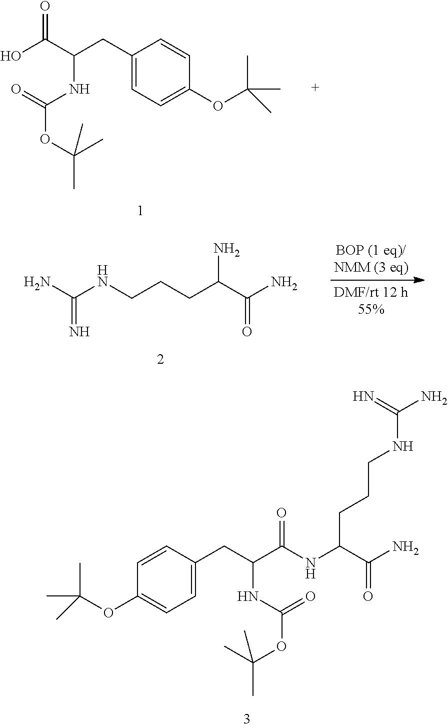

NMM (3.3 mL, 30 mmol) was added to a solution of Boc-Tyr(tBu)-OH (1) (3.374 g, 10 mmol) in DMF (40 mL), and the resulting mixture was stirred at room temperature for 1 h. Then, BOP (4.42 g, 10 mmol) and H-Arg-NH₂×2HCl (2) (2.46 g, 10 mmol) were added. The resulting reaction mixture was stirred overnight at room temperature. Upon completion of the reaction (TLC monitoring), the reaction mixture was filtered. The resulting solution was diluted with ethyl acetate (100 mL), washed with saturated sodium bicarbonate (3×50 mL), water (100 mL), 1M aqueous potassium hydrogenosulfate (3×50 mL), and brine (50 mL). The organic layer was dried over magnesium sulfate, filtered and concentrated in vacuo to afford compound 3 (2.7 g, 55% yield) as a colourless solid. The structure of compound 3 was confirmed by ¹H-NMR.

### Synthesis of Tyr(tBu)-Arg-NH₂ (HCl) (4)

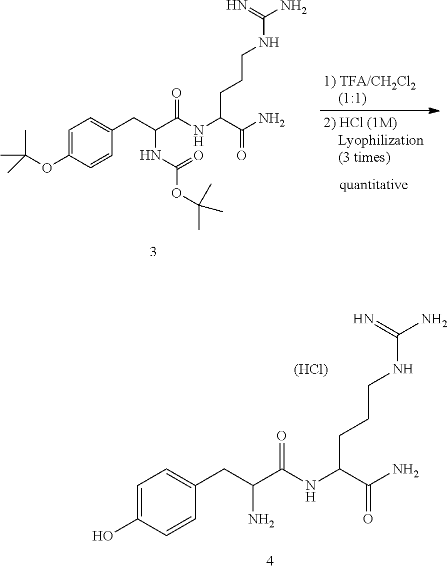

Compound 3 (2.6 g, 5.28 mmol) was dissolved in CH₂Cl₂ (8 mL) and the solution was cooled in an ice bath. TFA (8 mL) was added dropwise and the resulting mixture was stirred at 0° C. for 1-2 h. Upon completion of the reaction (TLC monitoring), the solvent was removed under reduced pressure. The resulting residue was triturated with ether, collected and dried in vacuo. The resulting white solid was dissolved in 1M aqueous HCl and lyophylized. This process was repeated three times to afford Tyr-Arg-NH₂ (4) (1.76 g, 100% yield) as a hydrochloride salt. The structure of compound 4 was confirmed by ¹H-NMR and HRMS (ESI).

### Synthesis of Ibu-Tyr-Arg-NH₂ (Ibu-KTP-NH2)

### Synthesis of Ibu-Tyr(tBu)-OMe (7)

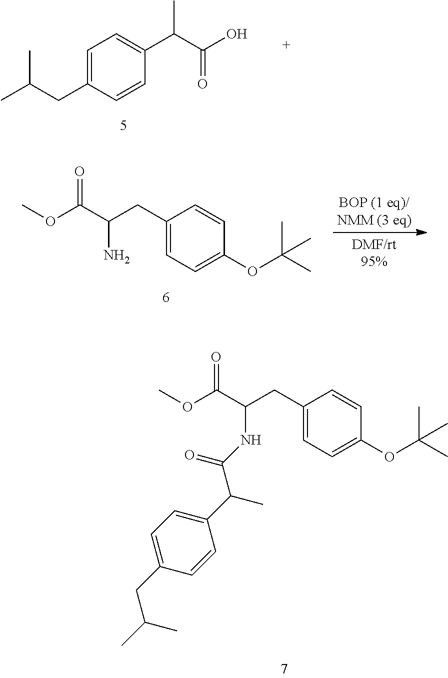

NMM (3.3 mL, 30 mmol) was added to a solution of (S)-Ibuprofen (5) (2.06 g, 10 mmol) in DMF (40 mL), and the resulting mixture was stirred at room temperature for 1 h. Then, BOP (4.42 g, 10 mmol) and H-Tyr(tBu)-OMe×HCl (6) (2.88 g, 10 mmol) were added. The resulting reaction mixture was stirred overnight at room temperature. Upon completion of the reaction (TLC monitoring), the solvent was removed in vacuo. The resulting residue was diluted with ethyl acetate (100 mL) washed with saturated sodium bicarbonate (3×50 mL), water (100 mL), 1M aqueous potassium hydrogenosulfate (3×50 mL), and brine (50 mL). The organic layer was dried over magnesium sulfate, filtered and concentrated in vacuo to afford compound 7 (4.17 g, 95% yield) as a colourless oil. The structure of compound 7 was confirmed by ¹H-NMR.

### Synthesis of Ibu-Tyr(tBu)-OH (8)

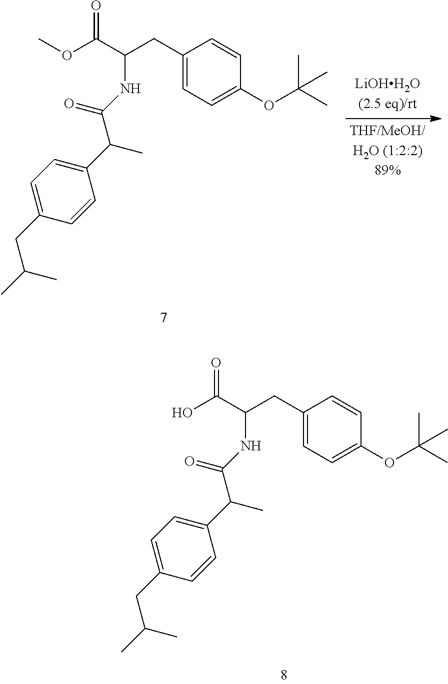

LiOH monohydrate (9.90 g, 23.62 mmol) was added to a solution of compound 7 (4.16 g, 9.45 mmol) in THF/MeOH/water (1:2:2, 78 mL), and the reaction mixture was stirred overnight at room temperature. The pH of the solution was adjusted to 2 by addition of 1M aqueous HCl. Then, the solution was extracted with CH₂Cl₂ (3×50 mL). The combined organic layers were dried over magnesium sulfate, filtered and concentrated in vacuo to afford Ibu-Tyr(tBu)-OH (8) (3.58 g, 89% yield) as a colourless solid. The structure of compound 8 was confirmed by ¹H-NMR and HRMS (ESI).

### Synthesis of Ibu-Tyr(tBu)-Arg-NH₂ (9)

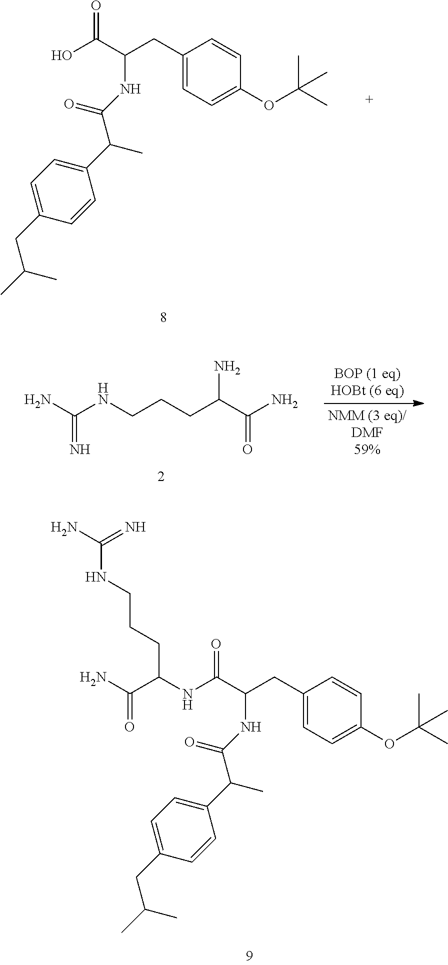

Ibu-Tyr(tBu)-OH (8) (3.5 g, 8.22 mmol) was dissolved in DMF (33 mL) and the solution was cooled at −15° C. H-Arg-NH₂×2HCl (2) (2.02 g, 8.22 mmol), HOBt (6.66 g, 49.32 mmol), BOP (3.63 g, 8.22 mmol), and NMM (2.71 mL, 24.66 mmol) were added. The resulting mixture was stirred at −15° C. for 1 h. After this time, the reaction mixture was warmed to room temperature and stirred for an additional 20 h. Upon completion of the reaction (TLC monitoring), the reaction mixture was diluted with ethyl acetate (100 mL). The resulting solution was washed with saturated sodium bicarbonate (3×50 mL), water (100 mL), 1M aqueous potassium hydrogenosulfate (3×50 mL), and brine (50 mL). The organic layer was dried over magnesium sulfate, filtered and concentrated in vacuo to leave compound 9 (2.81 g, 59% yield) as a colourless oil. The compound 9 was obtained and used as a diastereoisomeric mixture (dr=80%, HPLC). The structure of compound 9 was confirmed by ¹H-NMR.

### Synthesis of Ibu-Tyr-Arg-NH2 (HCl) (10)

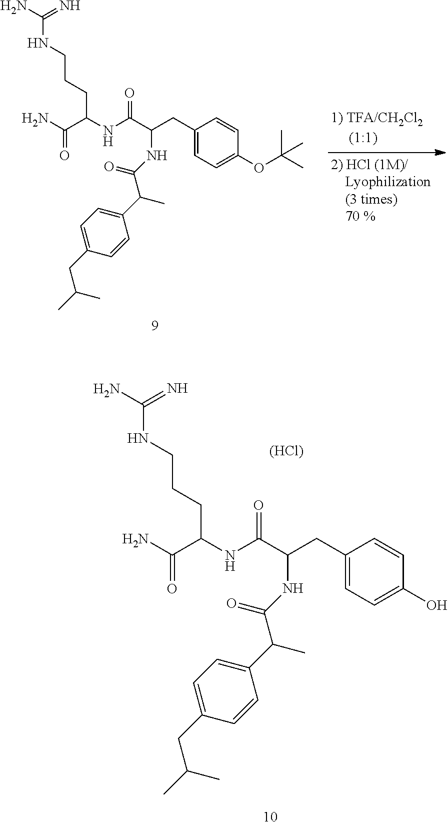

Compound 9 (2.75 g, 4.74 mmol) was dissolved in CH₂Cl₂ (7.10 mL) and the solution was cooled in an ice bath. TFA (7.10 mL) was added dropwise and the resulting mixture was stirred at 0° C. for 1-2 h. Upon completion of the reaction (TLC monitoring), the solvent was removed under reduced pressure. The resulting residue was triturated with ether, collected and dried in vacuo. The resulting white solid was dissolved in 1M aqueous HCl and lyophylized. This process was repeated three times to afford Ibu-Tyr-Arg-NH₂ (10) (1.74 g, 70% yield) as a hydrochloride salt. The structure of compound 10 was confirmed by ¹H-NMR and HRMS (ESI).

### Synthesis of Tyr-Arg-OH (KTP)

### Synthesis of Boc-Tyr(tBu)-Arg-OMe (12)

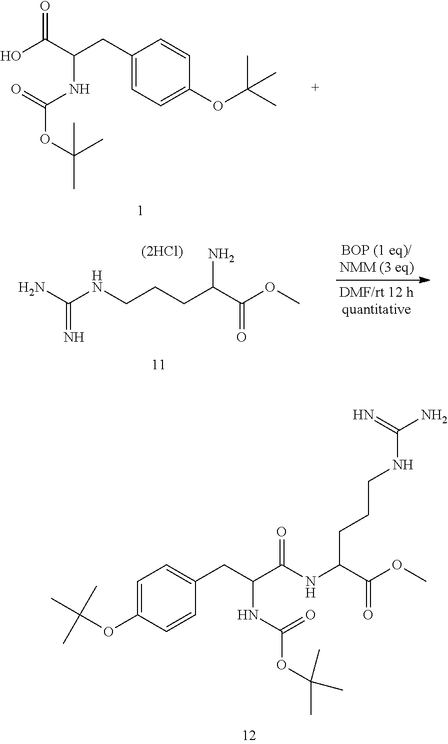

NMM (3.3 mL, 30 mmol) was added to a solution of Boc-Tyr(tBu)-OH (1) (3.374 g, 10 mmol) in DMF (40 mL) and the resulting mixture was stirred at room temperature for 1 h. Then, BOP (4.42 g, 10 mmol) and H-Arg-OMe×2HCl (11) (2.88 g, 10 mmol) were added. The resulting reaction mixture was stirred overnight at room temperature. Upon completion of the reaction (TLC monitoring), the reaction mixture was filtered. The resulting solution was diluted with ethyl acetate (100 mL) washed with saturated sodium bicarbonate (3×50 mL), water (100 mL), 1M aqueous potassium hydrogenosulfate (3×50 mL), and brine (50 mL). The organic layer was dried over magnesium sulfate, filtered and concentrated in vacuo to leave compound 12 (5.07 g, 100%) as a colourless solid. The structure of compound 12 was confirmed by ¹H-NMR and HRMS (ESI).

### Synthesis of Boc-Tyr(tBu)-Arg-OH (13)

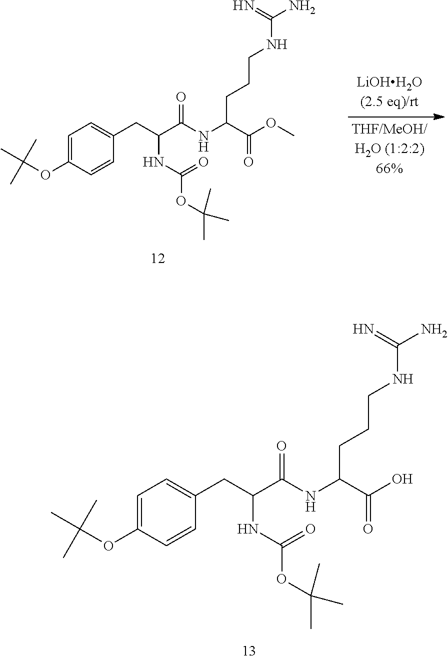

LiOH monohydrate (10.33 g, 24.62 mmol) was added to a solution of compound 12 (5.0 g, 9.85 mmol) in THF/MeOH/water (1:2:2, 82 mL), and the reaction mixture was stirred overnight at room temperature. Upon completation of the reaction (TLC monitoring), the organic solvents were removed under reduced pressure. The resultant aqueous solution was adjusted to pH 2 by addition of glacial acetic acid upon which the expected Boc-Tyr(tBu)-Arg-OH 13 was precipitated. The solid was collected by filtration, washed with cold water and dried in vacuo to afford compound 13 (3.21 g, 66% yield) as a colourless solid. The structure of compound 13 was confirmed by ¹H-NMR and HRMS (ESI).

### Synthesis of Tyr-Arg-OH(HCl) (14)

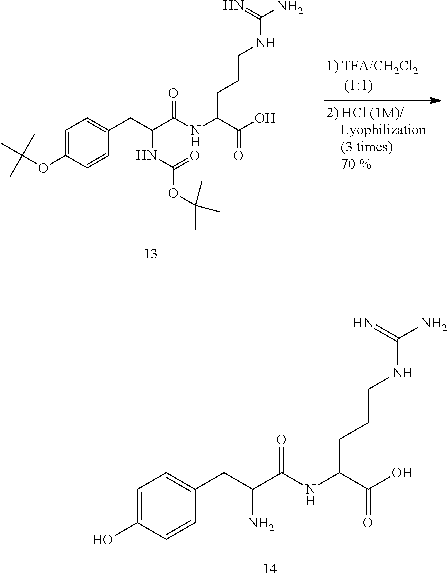

Compound 13 (3.10 g, 6.28 mmol) was dissolved in CH₂Cl₂ (9.5 mL) and the solution was cooled in an ice bath. TFA (9.5 mL) was added dropwise and the resulting mixture was stirred at 0° C. for 1-2 h. Upon completion of the reaction (TLC monitoring), the solvent was removed under reduced pressure. The resulting residue was triturated with ether, collected and dried in vacuo. The resulting white solid was dissolved in 1M aqueous HCl and lyophylized. This process was repeated three times to afford Tyr-Arg-OH (14) (2.03 g, 96% yield) as a hydrochloride salt. The structure of compound 14 was confirmed by ¹H-NMR and HRMS (ESI).

### Synthesis of Ibu-Tyr-Arg-OH (Ibu-KTP)

### Synthesis of Ibu-Tyr(tBu)-Arg-OMe (15)

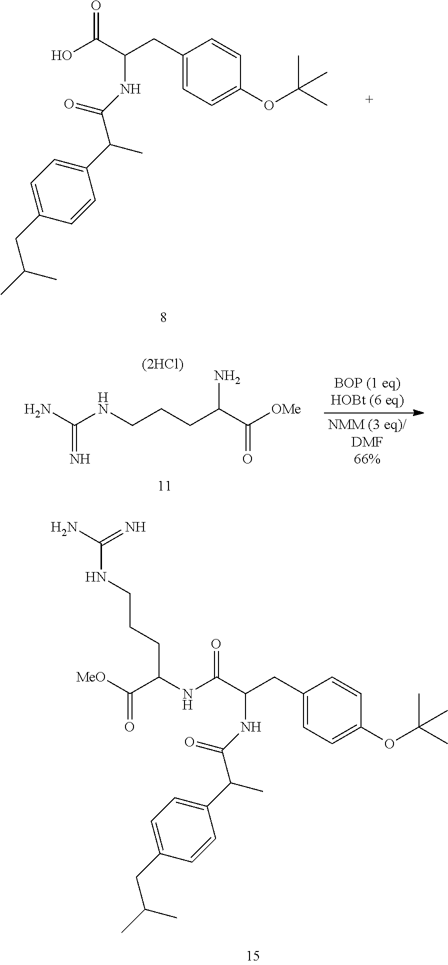

Ibu-Tyr(tBu)-OH (8) (3.5 g, 8.22 mmol) was dissolved in DMF (33 mL) and the solution was cooled at −15° C. H-Arg-OMe×2HCl (11) (2.152 g, 8.22 mmol), HOBt (6.66 g, 49.32 mmol), BOP (3.63 g, 8.22 mmol), and NMM (2.71 mL, 24.66 mmol), were added. The resulting mixture was stirred at −15° C. for 1 h. After this time, the reaction mixture was warmed to room temperature and stirred for an additional 20 h. Upon completion of the reaction (TLC monitoring), the reaction mixture was diluted with ethyl acetate (100 mL). The resulting solution was washed with saturated sodium bicarbonate (3×50 mL), water (100 mL), 1M aqueous potassium hydrogenosulfate (3×50 mL), and brine (50 mL). The organic layer was dried over magnesium sulfate, filtered and concentrated in vacuo to afford compound 15 (3.23 g, 66% yield) as a colourless oil. The compound 15 was obtained and used as a diastereoisomeric mixture (dr=80%, HPLC). The structure of compound 15 was confirmed by ¹H-NMR.

### Synthesis of Ibu-Tyr(tBu)-Arg-OH (16)

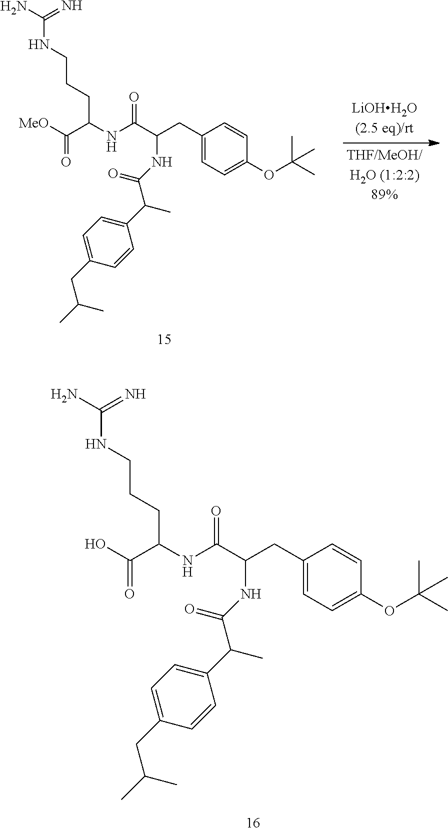

LiOH monohydrate (0.56 g, 13.42 mmol) was added to a solution of compound 15 (3.2 g, 5.37 mmol) in THF/MeOH/water (1:2:2, 44.5 mL), and the reaction mixture was stirred overnight at room temperature. Upon completation of the reaction (TLC monitoring), the organic solvents were removed under reduced pressure. The pH of the resulting aqueous solution was adjusted to 2 by addition of glacial acetic acid. Then, the solution was extracted with CH₂Cl₂ (3×40 mL). The combined organic layers were dried over magnesium sulfate, filtered and concentrated in vacuo to afford Ibu-Tyr(tBu)-Arg-OH 16 (2.78 g, 89% yield) as a colourless solid. The structure of compound 16 was confirmed by ¹H-NMR.

### Synthesis of Ibu-Tyr-Arg-OMe (17)

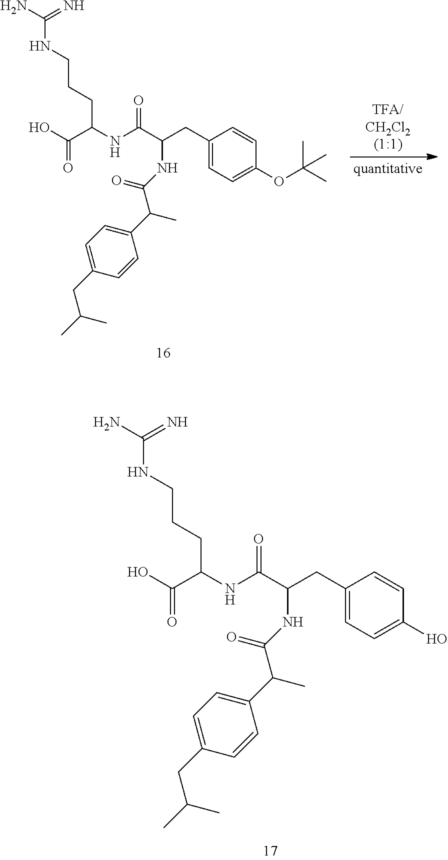

Compound 16 (0.4 g, 0.69 mmol) was dissolved in CH₂Cl₂ (1.0 mL) and the solution was cooled in an ice bath. TFA (1.0 mL) was added dropwise and the resulting mixture was stirred at 0° C. for 1-2 h. Upon completion of the reaction (TLC monitoring), the solvent was removed under reduced pressure. The resulting residue was triturated with ether, collected and dried in vacuo to afford Ibu-Tyr-Arg-OH 17 (0.36 g, 100% yield) as a colourless solid. The structure of compound 17 was confirmed by ¹H-NMR and HRMS (ESI).

### Solid Synthesis of KTP Derivatives:

Prior to the first aminoacid coupling, both swelling and Fmoc deprotection of the resin (Rink amide HBMA resin) are required. To accomplish this, the resin was left for 20 min in dichloromethane (DCM, 2 mL) and then 20 min in Dimethylformamide (DMF, 2 mL). The solution was removed by vacuum filtration being the resin treated with a solution of piperidine in DMF (3:7, 2.5 mL) for a total of 12 min. The resin was then filtrated again. For amino acid coupling, a solution in DMF (2.5 mL) of the Fmoc-protected aminoacid (3 eq) Ethyldiisopropylamine (DIEA, 3 eq) and O-Benzotriazole-N,N,N′,N′-tetramethyl-uronium-hexafluoro-phosphate, (HBTU, 3 eq) was added to the resin and left for 3 hours, with constant stirring. The Kaiser test was applied for evaluation of the coupling success. For the removal of the Fmoc group of the amino acid, piperidine in DMF was used as before. The second amino acid was coupled and deprotected using the conditions used for the first amino acid coupling, and the reaction was assessed by Kaiser test. To unlink the peptide from the resin, the reaction mixture was stirred for 2 hours in a solution of Trifluoroacetic acid (TFA):water:triisopropylsilane (TIS) (95:2.5:2.5, 2.5 mL) affording the free peptide. Between each step the reaction crude was washed with DCM and DMF (6×/1 min each). The solution was 3 times washed with Diethyl ether and centrifuged (5,000 rpm; 5 min). The precipitate obtained is the peptide. The precipitate was dissolved in water and lyophilized, obtaining each KTP derivative as a colourless solid (yield ranging between 60% and 80%). The purity of each peptide was analysed by HPLC.

| | 1^{st} amino acid | 2^{nd} amino acid |
|---|---|---|
| L-Tyr-D-Arg-NH₂ | D-Fmoc-Arg(Pmc)-OH | L-Fmoc-Tyr(tBu)—OH |
| D-Tyr-D-Arg-NH₂ | D-Fmoc-Arg(Pmc)-OH | D-Fmoc-Tyr(tBu)—OH |
| D-Tyr-L-Arg-NH₂ | L-Fmoc-Arg(Pmc)-OH | D-Fmoc-Tyr(tBu)—OH |
| Me-L-Tyr-L-Arg-NH₂ | L-Fmoc-Arg(Pmc)-OH | L-Fmoc—Me-Tyr(tBu)—OH |
| Me-L-Tyr-L-Arg-NH₂ | D-Fmoc-Arg(Pmc)-OH | L-Fmoc—Me-Tyr(tBu)—OH |

### ACRONYMS

BOP: benzotriazole-1-yl-oxy-tris-(dimethylamino)-phosphoniumfluorophosphate
DMF: N,N-dimethylformamide
dr: diastereoisomeric ratio
ESI: electrospray ionisation
NMM: N-methylmorpholine
¹H-NMR: Proton Nuclear Magnetic Resonance
HPLC: High Performance Liquid Chromatography
HRMS: High Resolution Mass Spectrometry
rt: room temperature
TFA: trifluoroacetic acid
TLC: Thin Layer Chromatography
HBMA: Hydroxy butyl methacrylate
Nr: Number

Our goal was to generate centrally-acting analgesic derivatives of KTP that are suitable for systemic administration. KTP is found in the central nervous system, in both brain and spinal cord, where it binds specific receptors and elicits strong analgesic effects. Accordingly, central administration of exogenous KTP produces strong analgesia. However, systemic administration of KTP has no relevant effect, as the molecule does not cross the blood-brain-barrier. The present invention relates to the development of KTP derivatives that cross the blood-brain-barrier and, therefore, can be used as central analgesics by systemic administration. We evidence that we have achieved this goal. First, the central analgesic action of compounds of the invention is shown by the result obtained for the Hot Plate test (a test suitable for identifying centrally and not peripherally acting analgesics). In addition, the formalin test showed an effect for compounds of the invention in both acute and tonic-chronic phases and the observed decrease in formalin-induced c-fos expression in the dorsal horn indicates that compounds of the present invention inhibit spinal nociceptive transmission, indicating a central action. Finally, the naloxone-reversible analgesic effect of compounds of the invention clearly supports a central mechanism of action for this molecule.

A further obstacle to the systemic administration of KTP is the rapid degradation of the molecule in contact with plasma proteases. The rapid plasma metabolization substantially reduces the amount of KTP in systemic circulation, creating an exposure deficit. In order to circumvent this problem, we generated derivatives of KTP-NH2 that are more resistant to plasma degradation. These degradation-resistant, blood-brain-barrier-permeable molecules of the invention are therefore amenable to systemic administration and have the capacity to penetrate into the central nervous system, where they exert a strong analgesic action.

The generated centrally-acting, degradation-resistant molecules of the invention can be administered systemically for the treatment of different types of pain. Pain, in general, may be divided into two subtypes: acute and chronic. Acute pain has a relatively short duration and a sudden onset. One type of acute pain, for example, is cutaneous pain felt on injury to the skin or other damaged tissues. Cutaneous nociceptors (pain-sensitive nerve endings) terminate just below the skin, and due to the high concentration of nerve endings, produce a well defined, localized pain of short duration. Chronic pain refers to a pain that persists after an acute injury, pain related to a persistent or degenerative disease and long-term pain from an unidentifiable cause, such as fibromyalgia. Common types for chronic pain include neuropathic pain, caused by damage to the nervous system such as diabetic neuropathies, inflammatory pain associated with arthritis and rheumatoid diseases, low back pain, cancer pain, post-operative pain and visceral pain.

1. A method of treating and/or prevention of pain in a subject comprising administering a composition of Formula (I) systemically, wherein the compound of formula (I) has the following structure: 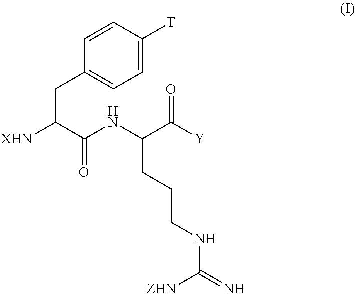
wherein X is hydrogen, R¹, R¹C(O), R¹CO₂, or a COX2 inhibitor,
wherein R¹ is C₁₋₂₀ alkyl, aryl, arylalkyl, alkyloxy or arylalkyloxy;
wherein Y is OR², NHR³, N(R³)₂, or a COX inhibitor;
wherein R² is hydrogen when X is a COX2 inhibitor, or R² is C₁₋₂₀ alkyl and each R³ is independently hydrogen or a C₁₋₄ alkyl;
wherein T is OR⁴, NNR⁵, N(R⁵)₂, or a COX inhibitor
wherein R⁴ is hydrogen or C₁₋₂₀ alkyl and each R⁵ is independently hydrogen or a C₁₋₄ alkyl;
wherein Z is hydrogen, R⁶, R⁶C(O), R⁶CO₂, or a COX2 inhibitor;
wherein R⁶ is C₁₋₂₀ alkyl, aryl, arylalkyl, alkyloxy or arylalkyloxy.

2. The method of claim 1, wherein X is hydrogen, or a COX2 inhibitor, Y is hydroxy, NH₂ or a COX inhibitor, Z is hydrogen and T is OH.

3. The method of claim 1 or claim 2, wherein the COX2 inhibitor is independently selected from ibuprofen, acetylsalicilic acid, meloxicam, valdecoxib, celecobix or refocobix.

4. The method of claim 1 or 2, wherein X and Z are hydrogen, T is OH and Y is NH₂ or wherein X is ibuprofen, Z is hydrogen, T is OH and Y is NH₂ or wherein X is ibuprofen, Z is hydrogen, T is OH and Y is OH, or wherein X is methyl, Z is hydrogen, T is hydroxy and Y is NH₂.

5. The method of claim 1 or 2, wherein the pain is chronic pain or acute pain.

6. (canceled)

7. A systemic pharmaceutical composition comprising a compound of formula (I) as defined in any one of claims 1 to 2 and a pharmaceutically acceptable excipient.

8. A method of treating and/or preventing pain in a subject comprising administering the systemic pharmaceutical composition as claimed in claim 7.

9. A method of preventing and/or treating pain comprising the administration to a patient in need thereof of a compound of formula (I), wherein said compound is administered systemically.

10. A compound selected from the group consisting of a compound of formula I, as defined in claim 1, wherein
X and Z are hydrogen, T is hydroxyl and Y is NH₂, in the form L-Tyrosyl-D-Arginine-NH₂ or D-Tyrosyl-D-Arginine-NH₂ or D-Tyrosyl-L-Arginine-NH₂; or
X is Ibuprofen, Z is hydrogen, T is hydroxyl and Y is NH₂ in the form Ibuprofen-L-Tyrosyl-L-Arginine-NH₂ or Ibuprofen-D-Tyrosyl-L-Arginine-NH₂, or
X is Ibuprofen, Z is hydrogen and T and Y are hydroxyl in the form Ibuprofen L Tyrosyl-L-Arginine OH, or
X is methyl, Z is hydrogen, T is hydroxyl and Y is NH₂, in the form methyl-L-Tyrosyl-L-Arginine-NH₂ or methyl-L-Tyrosyl-D-Arginine-NH₂, each optionally in the form of a pharmaceutical composition.
